(19) United States
(12) Patent Application Publication
(10) Pub. No.: US 2009/0117016 A1
(43) Pub. Date: May 7, 2009

(54) FUMIGATION AND SANITATION OF BIOLOGICAL PRODUCTS STORAGE SYSTEMS USING OZONE

(76) Inventors: R. SCOTT DECKER, CARTHAGE, MO (US); DALE A. ELTISTE, OVERLAND PARK, KS (US); RONALD T. NOYES, STILLWATER, OK (US)

Correspondence Address: POLSINELLI SHUGHART PC, 700 West 47th Street, Suite 1000, KANSAS CITY, MO, 64112, US

(21) Appl. No.: 12/240,727

(22) Filed: Sep. 29, 2008

Related U.S. Application Data
(60) Provisional application No. 60/976,164, filed on Sep. 28, 2007.

(51) Int. Cl. A61L 2/20; A23L 3/3418

(52) U.S. Cl. 422/186.08; 422/186.12

(57) ABSTRACT

An apparatus and method are provided for elimination of mold, pests, insects and the like from bulk, stored grain and milled grain products and other biological items by pushing an ozone-containing atmosphere up through the grain or biological items and providing and maintaining an ozone-containing atmosphere in contact with the upper surface of the grain or biological items and maintaining an ozone-containing atmosphere in the spaces of the storage contained that are below the grain or biological items.

### CROSS REFERENCE TO RELATED APPLICATION

This application claims priority under 35 U.S.C. 119(e) and 37 C.F.R. 1.78(a)(4) based upon copending U.S. Provisional Application Ser. No. 60/976,164 for FUMIGATION AND SANITATION OF BIOLOGICAL PRODUCTS STORAGE SYSTEMS USING OZONE, filed Sep. 28, 2007 which is incorporated herein by reference.

### FIELD OF THE INVENTION

The present invention relates to an apparatus and method of excluding and removing mold, pests, insects and the like from bulk, stored grain and milled grain products and other biological items. More particularly, the present invention first applies an ozone-containing atmosphere to the container intended to hold the grain or biological items to decontaminate the container followed by application of an ozone-containing atmosphere to the grain or biological items after they have been introduced into the container to remove mold, pests, insects and the like from the stored grain products or other biological items and followed by the maintaining of an ozone-containing atmosphere generally above and below the grain products and other biological items stored in the container to prevent a re-infestation by the mold, pests and/or insects.

### BACKGROUND OF THE INVENTION

When stored grains or bulk food products contain high moisture zones or if the air within the stored grains or bulk food products has humidity levels of approximately 70% or greater, mold may grow from mold spores trapped in the stored material. Mold growth is especially likely to occur when the bulk stored product is subjected to warmer temperatures, temperature in the range of 15 C to 25 C or higher. Such a moldy area of grain or other bulk food product is called “moisture migration”. “Moisture migration” is a major economic problem for the U.S. grain industry, for the rice industry, for producers of edible beans, for peanut warehouses, for seed stocks by large seed producers, and other biological granular products. Also, empty grain or food storage areas nevertheless often retain a minor amount of grain or food product and/or retain the odor of the food or grain product. Therefore, even an empty grain or food container attracts pests or may present a substrate for mold growth

### SUMMARY OF THE INVENTION

An apparatus and method of removing contaminants such as mold, pests, insects and the like in grain and bulk food products is provided comprising generating a gas or atmosphere comprising a low concentration of ozone in a carrier gas such a atmospheric air and applying the ozone-containing gas to the bottom of a container having a grain or food product therein. The ozone-containing gas is allowed to rise through the container contents and to fill the headspace of the container while an amount of ozone-containing gas is allowed to remain in the bottom of the container or the plenum area of chamber at the bottom of the container to prevent re-entry of mold, insects and pests from the bottom of the container or the plenum. Where necessary any roof eave spaces are filled to enable an ozone-containing gas atmosphere to be maintained in the headspace of the container to prevent re-entry of mold, insects and pests at the top of the container.

The ozone-containing gas may then be drawn off the headspace and allowed to enter a second container at the bottom to begin the decontamination of the second container in the manner described for the first container. The concentration of ozone in the ozone-containing gas is preferred to be in the range of 1 parts-per-million (ppm) to 15 ppm.

In one embodiment, excess ozone-containing gas is drawn off the headspace of the bin and the ozone converted to oxygen to prevent damage to the surrounding environment.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention.

### BRIEF DESCRIPTION OF THE DRAWING

Preferred embodiments of the invention, illustrative of the best modes in which the applicant has contemplated applying the principles, are set forth in the following description and are shown in the drawings and are particularly and distinctly pointed out and set forth in the appended claims.

FIG. 1 shows a schematic cross-section view of a grain storage elevator or bin 10 loaded with stored grain or food product 12 and showing the apparatus of the present invention installed;

FIG. 2 shows a schematic cross-section view of two grain storage bins or elevators loaded with grain and connection in a series for application of the present method to the two grain storage structures and having an ozone destruction unit at the exhaust area; and

FIG. 3 shows a schematic cross-section view of two grain storage bins or elevators loaded with grain and connection in a series for application of the present method to the two grain storage structures and showing an ozone recirculating apparatus in combination therewith.

### DESCRIPTION OF THE PREFERRED EMBODIMENTS

As required, detailed embodiments of the present inventions are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

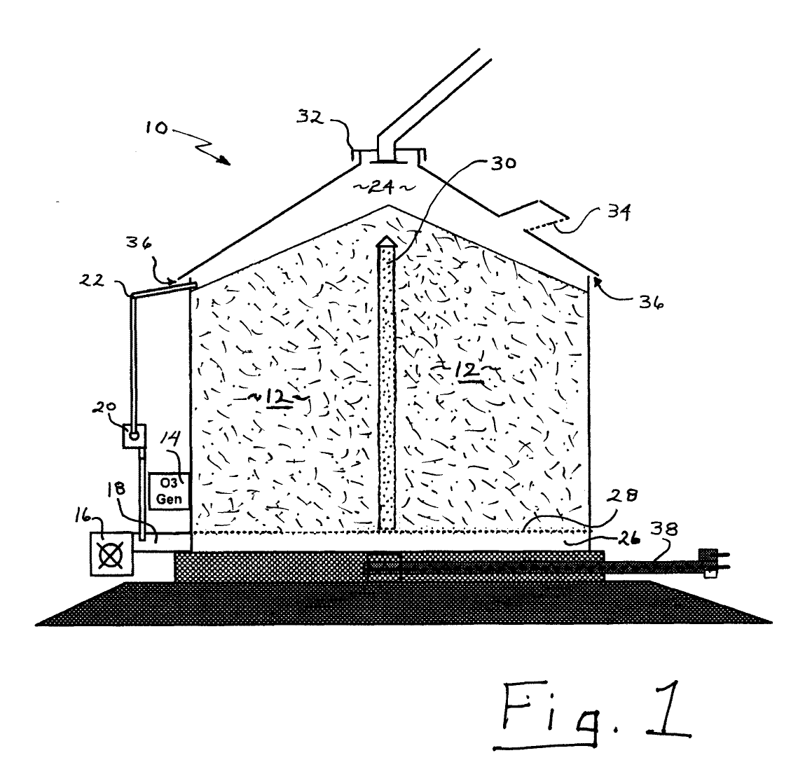

In FIG. 1 is shown the apparatus used in the method for treating empty and filled grain and food product storage devices with an ozone atmosphere to sanitize, control and protect the storage container 10 and stored grain or food products 12 from microbial and biological pests. Such microbial and biological pests can include, at least, molds, fungus, insects, birds, rodents, moisture migration, and other unsanitary elements which are detrimental to the quality of the grain and food products. The ozone treatment is supplied by on-site ozone generators 14 which are connected to the storage container 10 to communicate to the storage container 10 the ozone and/or ozone/air mixture. The ozone generator 14 can be operated either continuously or intermittently as needed. The ozone is supplied from the ozone generator 14 in a required amount according to the selected treatment process to accomplish the specific fumigation or sanitation process desired by the product storage facility manager or representative.

Still referring to FIG. 1, in general, the ozone delivery system includes an ozone generator 14 capable of delivering substantially high levels of ozone ranging from at least 100 ppm to 3000 ppm. In the method taught herein, the produced ozone can be supplied to empty storage containers 10 as ozone or as a combination or an ozone and air atmosphere for the initial purpose of rapidly sanitizing one or more empty storage containers 10. The method also includes supplying the produced ozone or ozone and air atmosphere to kill or remove molds, mildew, off odors, and pests in the grain or food product 12 in a filled storage container 10. In an additional application of the device, it should be appreciated that the ozone generator 14 also is capable of delivering low levels of ozone, generally in the range of 1 part-per million (ppm) to 15 ppm, for the purpose of providing economical and prophylactic protection from pests and molds and mildew and the like of either an empty storage container 10 or a filled storage container 10. In such cases, the pests may initially attempt to enter the container, or begin growth, but either will be killed by the ozone-containing atmosphere or the pest will find the encountered ozone-containing atmosphere unpleasant and retreat from the container.

In the embodiment of FIG. 1, an ozone generator 14 is connected to a storage container 10. The ozone gas and/or an ozone and air gas mixture from the ozone generator 14 is propelled into the container 10 by an aeration fan or blower 16 that is positioned to deliver the ozone gas and/or an ozone and air gas mixture from the ozone generator 14 via a connection 18 to the base of the container 10. The ozone generator 14 also is connected to a small fan or blower 20 that can provide closed loop recirculation of the ozone gas and/or an ozone and air gas mixture via recirculation pipe 22. The small fan 20 also can be used to directly deliver the ozone gas and/or an ozone and air gas mixture directly to headspace 24 of the container 10. The ozone gas and/or an ozone and air gas mixture that is delivered to the base of the container 10 is, in the embodiment shown in FIG. 1, directed into the plenum 26 of storage container 10 that is below a perforated floor 28 in the bottom of storage container 10. In the embodiment of FIG. 1, a perforated distribution pipe or pipes 30 can be connected to plenum 26 to assist in distributing the ozone gas and/or an ozone and air gas mixture into the stored grain or food product 12 stored in container 10. The deliver of the ozone gas and/or an ozone and air gas mixture to headspace 24 is assisted by sealing the roof fill cap 32 and the roof exhaust vent 34 and sealing the roof eave gaps 36 of the container 10.

As shown in FIG. 1 the deliver of ozone gas and/or an ozone and air gas mixture to storage container 10 via the entry and exit areas of the container including connections 18 and 22 provides ozone gas and/or an ozone and air gas mixture to the storage container headspace 24 (the area above the stored contents of the container), the base air duct 18 and/or the plenum space 26 at the bottom of the container 10 and to the stored grain or food product of the container 10 via the distribution pipe 30 and the perforated floor 28 above the plenum 26 of container 10. It will be appreciated that a typical grain bin or bulk food storage container 10 is provided with a variety of means of adding or evacuating liquids or gases to or from the contents of the storage container 10. All of the means can be used to add the ozone gas and/or an ozone and air gas mixture to the container 10 and/or its stored grain or food product 12. Such means of adding or evacuating may take the form of a perforated floor section 28 and/or an aeration duct system 38 and/or an under floor plenum 26, and/or an on-floor perforated aeration duct means, and/or an in-floor aeration duct means, or other variations means for introducing liquids or gases to or from the contents of the storage container.

In operation of the method of the embodiment of FIG. 1, the ozone gas and/or an ozone and air gas mixture is introduced into storage container 10 through any one or more of the above-listed structures including the gas recirculation pipe 22, the plenum 26, the distribution pipe 30, the perforated floor section 28 the aeration duct system under the floor plenum 38, an on-floor perforated aeration duct means, or an in-floor aeration duct means, or other variations means for introducing liquids or gases to or from the contents of the storage container. However, it is preferred to introduce the ozone gas and/or an ozone and air gas mixture at the bottom of the container 20 either through the pipes in the bottom of the container 20 or through the plenum area 26 just below the bottom of the container. Introduction of the ozone gas and/or an ozone and air gas mixture through the plenum 26 allows the ozone gas and/or an ozone and air gas mixture to move upwardly through the stored grain or food product 12 in the container 10. This allows entry of the ozone gas and/or an ozone and air gas mixture into the lower areas of the storage container 10 and entry into the stored grain or food product 12 of the storage container to achieve wide distribution of the ozone gas and/or an ozone and air gas mixture across the bottom of the stored grain or food product 12 held in the storage container 10. Upon entry, the ozone-container gas or atmosphere penetrates upwardly into the product mass held in the container for a substantial distance above the structural floor of the container.

The apparatus and method present a protection process that provides a layering or blanket of ozone-containing gas that is substantially over and within the stored grain or food product 12 surfaces. The ozone-containing gas, at the same time, may be allowed to permeate the headspace 24 volume of the container. As the ozone-containing gas contacts the upper or top surface of the stored grain or food product 12 in the container 10, the ozone-containing gas migrates downwardly, into the stored grain or food product 12 of the container 10, for a substantial distance below the top surface. This ozone blanket in the headspace 24 shields the upper portion of the stored grain or food product 12 from entry by pests. In addition the entry of the ozone-containing gas through the plenum 26 allows the ozone-containing gas to migrate upwardly from the bottom of the stored grain or food product 12 while one or more distribution pipes 30 that extend into the mass of the stored grain or food product 12 allow the delivery of ozone-containing gas of a desired concentration generally into the center of the stored grain or food product 12.

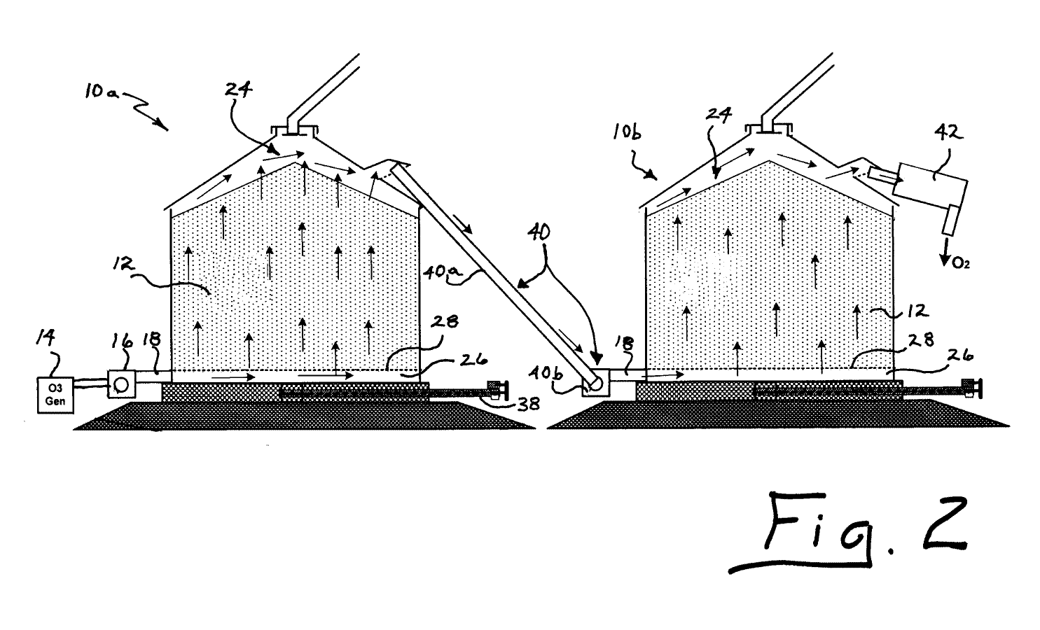

In FIG. 2, an alternate embodiment is shown that includes a gas conveyance system or means 40 comprising connective tubing 40a and a blower 40b for communicating or transmitting the ozone gas and/or an ozone and air gas mixture from generator 14 to several locations of storage containers 10b. In the operation of the embodiment of FIG. 2 the ozone gas and/or an ozone and air gas mixture is first communicated to a first storage container 10a for treatment of the grain or food product 12 contained in the first storage container 10a. Subsequently, the ozone gas and/or an ozone and air gas mixture introduced into the first storage container 10a is communicated to a second storage container 10b via the conveyance means 40 comprising connective tubing 40a and a blower 40b. This alternate embodiment can provide ozone-containing gas into the interior of a volume of grain or food product stored in the container. If desired, the alternate embodiment of FIG. 2 also can provide the communicated ozone gas and/or ozone and air gas mixture from container 10a to the headspace area 24 of container 10b by the addition of a pipe similar to pipe 22 of FIG. 1 that travels from connective tubing 40a to headspace 24 of container 10b. It will be appreciated that multiple, containers 10a, 10b, 10c, and 10d can be connected in the above-described manner to allow transfer of ozone-containing gas among the various containers 10a, 10b, 10c, and 10d. Also present in the system is an ozone destroying unit 42 that eliminates ozone in the gas passing out of the system. Typically, the ozone destruction unit operates by catalysis of the ozone into oxygen. The ozone-containing gas enters the ozone destruction unit and a blower in the unit draws in fresh air to mix with the ozone-containing gas. The fresh air and ozone mixture may then be passed through a heating chamber to reduce ambient moisture to increase catalyst efficiency. The heated fresh air and ozone mixture proceeds to a catalytic chamber where the ozone is catalyzed in oxygen.

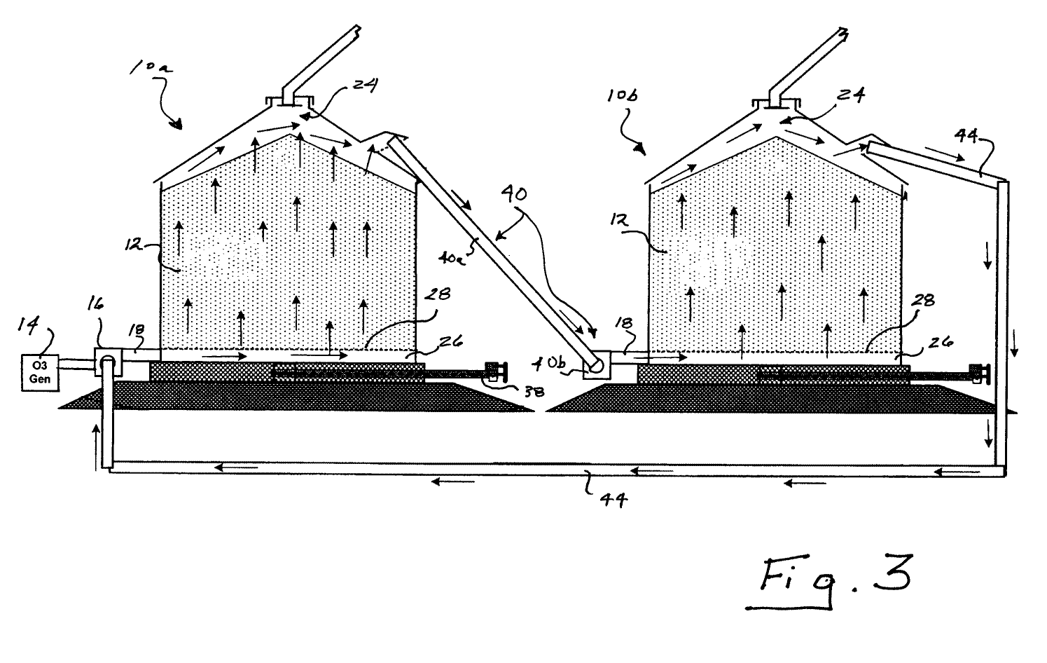

As shown in FIGS. 2 and 3, the gas conveyance system or means 40 is capable of slowly moving a volume of ozone-containing gas that will displace the air volume within the grain or food product 12 at least once per day, approximately, for thereby providing a generally uniform or “homogenized” atmosphere of distributed ozone gas throughout the container 10 structures and generally uniformly throughout the grain or food product 12 within the containers 10. In contrast, the single container, internal gas, and optionally, recirculating ozone-containing gas moving system of FIG. 1 provides a benefit and purpose of maintaining a substantially uniform air and product temperature throughout the product mass by allowing the recirculating of the ozone gas and/or an ozone and air gas mixture while allowing the addition of additional atmospheric air via fan 16 and additional ozone gas and/or an ozone and air gas mixture from generator 14 to moderate the temperature of the recirculating ozone gas and/or an ozone and air gas mixture in container 10. The internal gas moving system also serves to maintain a substantially uniform moisture content throughout the grain or food product 12. This is achieved as the moving ozone-containing gas air will absorb moisture from a first portion of the grain or food product 12 and re-distribute the moisture by allowing reabsorption of moisture in a second area of the grain or food product 12.

Referring now to FIG. 2, the embodiment that allows the transfer of the ozone gas and/or an ozone and air gas mixture from a first container 10a to a second container 10b will be described in detail. Containers 10a, 10b may or may not have a grain or food product within during the operation of the method. Initially, a conveyance means 40 comprising connective tubing 40a and a blower 40b is provided between containers 10a and 10b to allow transfer of gaseous atmosphere between containers 10a, 10b. The operation is initiated by the generation of ozone by ozone generator 14. The ozone gas and/or an ozone and air gas mixture generated is then passed into blower 16 which communicates the ozone gas and/or an ozone and air gas mixture into plenum 26. From plenum 26 the ozone gas and/or an ozone and air gas mixture is able to rise through perforated floor 28 into container 10a. The contact of the ozone gas and/or an ozone and air gas mixture with container 10a and/or with the grain or food product 12 which may be within container 10a accomplishes the killing of the pests and/or mold that may be in the container 10a and/or the grain or food product 12 stored in container 10.

As the ozone gas and/or an ozone and air gas mixture rises through container 10a it arrives at headspace 24 and the ozone gas and/or an ozone and air gas mixture kills and pests and/or mold that may be present in headspace 24. As the ozone gas and/or an ozone and air gas mixture arrives in headspace 24 is begins to be drawn out of headspace 24 and into a conveyance means 40 comprising connective tubing 40a and a blower 40b that connects the atmosphere of container 10a with the entry points into container 10b. The ozone gas and/or an ozone and air gas mixture is pulled into conveyance means 40 by blower 40b and pushed into plenum 26 of container 10b. Once in plenum 26 the ozone gas and/or an ozone and air gas mixture rises through perforated floor 28 of container 10b and kills any pests and mold in container 10b. The ozone gas and/or an ozone and air gas mixture rises to fill headspace 24 of container 10b and to kill any pests and mold in headspace 24. The ozone gas and/or an ozone and air gas mixture the moves into ozone destroying unit 42 where the remaining ozone is reduced to oxygen for release into the external atmosphere.

Referring now to FIG. 3 the system generally described in FIG. 2 is shown having the addition of a recirculation tube 44 in the place of ozone destroying unit 42 (FIG. 2). In this embodiment the ozone gas and/or an ozone and air gas mixture rather than being passed into ozone destroying unit 42. In this manner the benefits previously described for the recirculation of the ozone gas and/or an ozone and air gas mixture in the embodiment of FIG. 1 may be achieved in the embodiment having multiple containers 10a, 10b.

It will be appreciated that the ozone gas and/or an ozone and air gas mixture delivery apparatus and method process can kill or driving out of insect pests, rodents, birds, and biological microbial spores, fungus, molds, toxins, and other storage pests from the container and from the stored material. The ozone-containing gas, ozone-blanketing system can be started in an empty container 10 as a sanitizing process and can be continued during the filling of the container 10 with the grain or food product 12. The method can be operated to supply a substantially continuous flow or an intermittent, controlled flow of an economical low level of ozone-containing gas concentration, such as from 1 ppm to 10 ppm, or higher as desired. A low concentration level of ozone concentration can be maintained economically to provide continuous protection of the grain or food product 12 from the start of container filling through filling completion. In this manner a complete barrier layer of ozone-containing gas is provided which surrounds and permeates the stored product, thereby protecting its quality from entry of pests.

By use of the described method the stored product 12 is maintained at a very high level of quality from harvest to market delivery. Damage to the stored grain or food product 12 from mold, insects or other pests is avoided. The stored grain and food product 12 will not have insect damaged kernels (IDK), or live or dead insects, or off odor from molds, and will avoid disqualifying defects other than normal dockage and foreign materials from defects that were previously existing in the grain or food product at the time it was transferred into storage.

Another method embodiment of the invention uses ozone that is generated by use of prepared dry air or a high oxygen content air stream on a much larger scale. In this embodiment, the ozone-containing gas is diluted with air. When oxygen is used to generate the ozone fail-safe procedures known to those skilled in the art are provided to insure that the oxygen concentration cannot exceed 30% in the agricultural product being treated. This embodiment allows the re-claiming, or improving, the marketability and value of an already negatively affected stored grain or bulk food product by destroying detrimental microbiological contamination or pestilence that is causing damage to the grain of stored product by contaminants as aflotoxin, vomitoxin, pestilence and the like. This embodiment also provides off-odor removal from the grain or food product 12 where the off-odor is a result of mold and mildew and/or fungus and the like. In operation, an ozone-containing gas, having ozone at a relatively high concentration such as approximately 15% by weight to a maximum of 34% by weight ozone, is introduced into a container 10 as described above. The ozone is communicated into the bottom of the stored grain or food product 12 at a sufficient flow rate to contact the product in every available space of storage container 10. This is introduction of the ozone gas and/or an ozone and air gas mixture is continued until effective concentrations of ozone are detected at the top or the headspace 24 of the container 10 holding the stored grain or food product. The relatively high concentration of ozone-containing gas of between approximately 15% by weight to a maximum of 34% by weight ozone is then maintained for a sufficient period of time to achieve the desired disinfection or fumigation result. In a preferred embodiment an ozone concentration of approximately thirty-four percent (34%) by weight is preferred. Subsequently, as shown in FIGS. 2 and 3, these high concentrations of ozone-containing gas coming from the headspace 24 can be re-directed to adjacent storage bins, and/or re-circulated back to the bottom or destroyed (FIG. 2) depending on the situation encountered. This embodiment provides an opportunity to raise the value of a stored product and can be used in conjunction with the other embodiments to protect or reclaim agricultural products from several bioterrorism risks associated with our current world threats and difficulties.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Certain changes may be made in embodying the above invention, and in the construction thereof, without departing from the spirit and scope of the invention. It is intended that all matter contained in the above description and shown in the accompanying drawings shall be interpreted as illustrative and not meant in a limiting sense.

Having now described the features, discoveries and principles of the invention, the manner in which the inventive method and apparatus for removing and excluding mold, insects, and pests and the like from grain and bulk food products are constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

I claim:

1. An apparatus for fumigating containers comprising:
an ozone generator for providing an efficacious amount of ozone gas
a container having an interior area, and
a blower operatively connected between said ozone generator and said container to introduce said ozone gas into said container.

2. The apparatus of claim 1 wherein said efficacious amount of ozone gas is between one (1) parts per million and fifteen (15) parts per million.

3. The apparatus of claim 1 wherein said efficacious amount of ozone gas is between fifteen (15) percent by weight and thirty-four (34) percent by weight.

4. The apparatus as claimed in claim 1 further comprising a plenum at the base of said container said plenum in gaseous communication with said container interior area said plenum receiving said ozone gas from said blower prior to introduction of said gas into said container.

5. The apparatus as claimed in claim 4 further comprising a perforated container floor between said plenum and said container for communication of said ozone gas from said plenum to said container.

6. The apparatus as claimed in claim 1 further comprising a distribution pipe extending into said container for communication of ozone into said container interior.

7. The apparatus as claimed in claim 4 further comprising a distribution pipe extending from said plenum into said container for communication of ozone into said container interior.

8. The apparatus as claimed in claim 1 further comprising a pipe extending to a container headspace from said blower to introduce ozone gas into headspace.

9. The apparatus as claimed in claim 1 further comprising means for transferring said ozone gas from a headspace of a first container to a second container for introduction of said ozone gas into said second container.

10. The apparatus as claimed in claim 9 wherein said means for transferring comprises a transfer tube and a blower operatively connected to said second container to introduce said ozone gas into said second container.

11. The apparatus as claimed in claim 1 further comprising an ozone destroying device connected to an outlet from said container.

12. The apparatus as claimed in claim 4 further comprising a recirculation tube for communicating ozone gas between a container headspace and said plenum.

13. The apparatus as claimed in claim 12 further comprising a blower attached to said recirculation tube.

14. A storage container fumigation apparatus comprising:
a container having an interior area and a headspace area and a gas permeable floor positioned above a plenum said gas permeable floor allowing gas migration between said plenum and said container interior area and said headspace area,
a gas distribution pipe extending from said plenum to said interior area for communication of gas from said plenum to said interior area,
an ozone generator for providing an efficacious amount of ozone gas, and
a blower operatively connected between said ozone generator and said container to introduce said ozone gas into said plenum.

15. The apparatus of claim 14 wherein said efficacious amount of ozone gas is between one (1) parts per million and fifteen (15) parts per million.

16. The apparatus of claim 14 wherein said efficacious amount of ozone gas is between fifteen (15) percent by weight and thirty-four (34) percent by weight.

17. The apparatus as claimed in claim 14 further comprising a pipe extending to a container headspace from said blower to introduce ozone gas into headspace

18. The apparatus as claimed in claim 14 further comprising means for transferring said ozone gas from a headspace of a first container to a second container for introduction of said ozone gas into said second container.

19. The apparatus as claimed in claim 18 wherein said means for transferring comprises a transfer tube and a blower operatively connected to said second container to introduce said ozone gas into said second container.

20. The apparatus as claimed in claim 14 further comprising an ozone destroying device connected to an outlet from said container.

21. The apparatus as claimed in claim 14 further comprising a recirculation tube for communicating ozone gas between said headspace and said plenum.

22. The apparatus as claimed in claim 21 further comprising a blower attached to said recirculation tube.
